(19) United States
(12) Patent Application Publication
(10) Pub. No.: US 2008/0071357 A1
(43) Pub. Date: Mar. 20, 2008

(54) Controlling biodegradation of a medical instrument

(76) Inventors: Timothy S. Girton, Edina, MN (US); Daniel J. Gregorich, St. Louis Park, MN (US); Todd Messal, Plymouth, MN (US)

Correspondence Address: FISH & RICHARDSON PC, P.O. BOX 1022, MINNEAPOLIS, MN, 55440-1022, US

(21) Appl. No.: 11/893,175

(22) Filed: Aug. 15, 2007

Related U.S. Application Data
(60) Provisional application No. 60/826,002, filed on Sep. 18, 2006.

(51) Int. Cl. A61F 2/82

(52) U.S. Cl. 623/1.16; 623/1.15; 623/1.49

(57) ABSTRACT

An endoprothesis comprising a bioerodible body having local erosion rates of the body that vary as a continuous function of radial distance from the longitudinal axis.

### CROSS-REFERENCE TO RELATED APPLICATIONS

This application claims priority under 35 USC §119(e) to U.S. Provisional Patent Application Ser. No. 60/826,002, filed on Sep. 18, 2006, the entire contents of which are hereby incorporated by reference.

### TECHNICAL FIELD

This invention relates to bioerodible endoprostheses.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

It is sometimes desirable for an implanted endoprosthesis to erode over time within the passageway. For example, a fully erodible endoprosthesis does not remain as a permanent object in the body, which may help the passageway recover to its natural condition. Erodible endoprostheses can be formed from, e.g., a polymeric material, such as polylactic acid, or from a metallic material, such as magnesium, iron or an alloy thereof.

### SUMMARY

In one aspect, an endoprothesis includes a bioerodible body having local erosion rates of the body that vary as a continuous function of radial distance from the longitudinal axis.

In one aspect, an endoprothesis can include a bioerodible member having a solid cross-section with an arcuate outer surface.

Embodiments of these aspects can include one or more of the following features.

A first portion of the body can have a first erosion rate and a second portion of the body can have a second erosion rate that is greater than the first erosion rate and the distance between the second portion of the body and the longitudinal axis can be greater than the distance between the first portion of the body and the longitudinal axis. A first portion of the body can have a first erosion rate and a second portion of the body has a second erosion rate that is less than the first erosion rate and the distance between the second portion of the body and the longitudinal axis is greater than the distance between the first portion of the body and the longitudinal axis.

The endoprosthesis can define a tubular lumen parallel to the longitudinal axis.

The body can include a polymer. In some instances, the body can include a cross-linkable polymer that has a degree of cross-linking that varies as a function of radial distance from the longitudinal axis.

The body can include at least one metal and, in some instances, can also include at least one polymer.

A first erosion rate of a first portion of the body (e.g., a bioerodible member) can be between about 1 and 3 percent of the mass of the first portion per day (e.g., between about 0.1 and 1 percent of the mass per day). In some instances, a second erosion rate of a second portion of the body can be between about 0.1 and 1 percent of the mass of the second portion per day.

The endoprothesis can include a stent.

The bioerodible member can include a substantially round portion. In some instances, an endoprothesis can also include a plurality of bioerodible members (e.g., members including bioerodible wire) attached together, each of the bioerodible members having substantially round solid cross-sections.

The outer surface of the bioerodible member can include flat faces joined by radiused transition sections.

In one aspect, an endoprothesis can include a body having local erosion rates that vary along a first direction, and that vary along a second direction.

The endoprothesis can have a longitudinal axis, the first direction is transverse to the longitudinal axis, and the second direction is along the longitudinal direction.

Embodiments may include one or more of the following advantages. The endoprostheses may not need to be removed from a lumen after implantation. The endoprostheses can have a low thrombogenecity and high initial strength. The endoprostheses can exhibit reduced spring back (recoil) after expansion. Lumens implanted with the endoprostheses can exhibit reduced restenosis. The rate of erosion of different portions of the endoprostheses can be controlled, allowing the endoprostheses to erode in a predetermined manner, reducing, e.g., the likelihood of uncontrolled fragmentation. For example, the predetermined manner of erosion of the endoprosthesis can be from an inside surface to an outside surface, from an outside surface to an inside surface, from a first end of the endoprosthesis to a second end of the endoprosthesis, or from both the first and second ends of the endoprothesis.

Erosion or bioerosion as described herein includes dissolution, degradation, absorption, corrosion, resorption and/or other disintegration processes in the body. A bioerodible material or device is a material or a device that a user expects to erode over a certain timeframe (which can be defined by a manufacturer of the material or the device). Erosion is an intended and desirable process. In some embodiments, a bioerodible material or device loses more than about 80% of the mass of the largest remaining portion of the initial material or device over one year, or more than about 99% over two years. In contrast, for a non-bioerodible material or device, erosion is an unintended and undesirable event.

Erosion rates can be measured with a test endoprosthesis suspended in a stream of Ringer's solution flowing at a rate of 0.2 m/second. During testing, all surfaces of the test endoprosthesis can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter. As used herein, local erosion rates indicate the erosion rate of a stent at a specific position on the stent.

As used herein, an “alloy” means a substance composed of two or more metals or of a metal and a nonmetal intimately united, for example, by being fused together and dissolving in each other when molten.

All publications, patent applications, patents, and other references mentioned herein are incorporated by reference herein in their entirety.

The details of one or more embodiments of the invention are set forth in the accompanying drawings and the description below. Other aspects, features, and advantages will be apparent from the description and drawings, and from the claims.

### DESCRIPTION OF DRAWINGS

FIG. 1A is a perspective view of an embodiment of an erodible stent; and FIG. 1B is a cross-sectional view of the stent of FIG. 1A, taken through section 1B-1B.

FIGS. 2-4 illustrate erosion of an erodible stent within a body passageway.

FIGS. 5-8 are cross-sectional views of embodiments of an erodible stent.

FIGS. 9A and 9B are, respectively, perspective views of a polymer sheet and a stent formed from the polymer sheet.

FIG. 10A is a perspective view of an embodiment of an erodible stent; and FIG. 10B is a cross-sectional view of a portion of the stent of FIG. 10A, taken along line 10B-10B.

FIGS. 11 and 12 are side views of embodiments of erodible stents.

### DETAILED DESCRIPTION

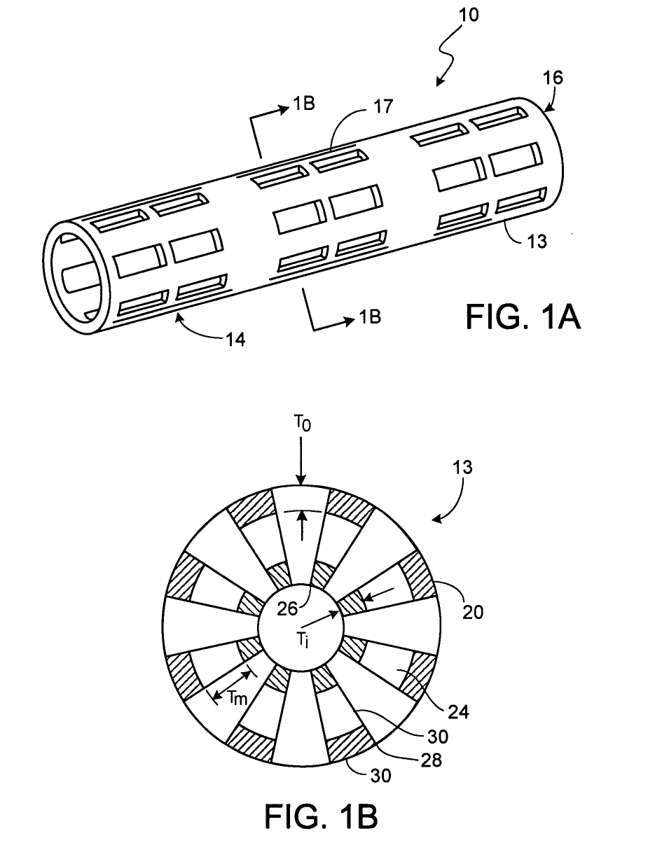

FIGS. 1A and 1B show an erodible endoprotheses (as shown, stent 10) configured to erode in a controlled and predetermined manner. As shown, stent 10 includes a tubular body 13 having an outer portion 20, an inner portion 26, and middle portion 24 between the outer and inner portions. Outer portion 20 includes a first metallic composition, such as an erodible magnesium alloy, that has a first erosion rate. Middle and inner portions 24, 26 include second and third metallic compositions that, respectively, have second and third erosion rates. The third erosion rate is lower than the second erosion rate and the second erosion rate lower than the first erosion rate. For example, the second and third compositions can include the magnesium alloy of outer portion 20 containing magnesium nitride (e.g., Mg₃N₂), which is relatively stable against corrosion and can reduce the erosion rate of the magnesium alloy. Alternatively or additionally, without wishing to be bound by theory, it is believed that the reduction in corrosion can also be due to the densification of the magnesium alloy as a result of nitrogen bombardment. As a result, without substantially changing the bulk mechanical properties of stent 10, middle and inner portions 24, 26 can extend the time it takes the stent to erode to a particular degree of erosion, relative to a stent including the magnesium alloy without the magnesium nitride. This extension of time allows cells of the passageway in which stent 10 is implanted to better endothelialize around the stent, for example, before the stent erodes to a degree where it can no longer structurally maintain the patency of the passageway.

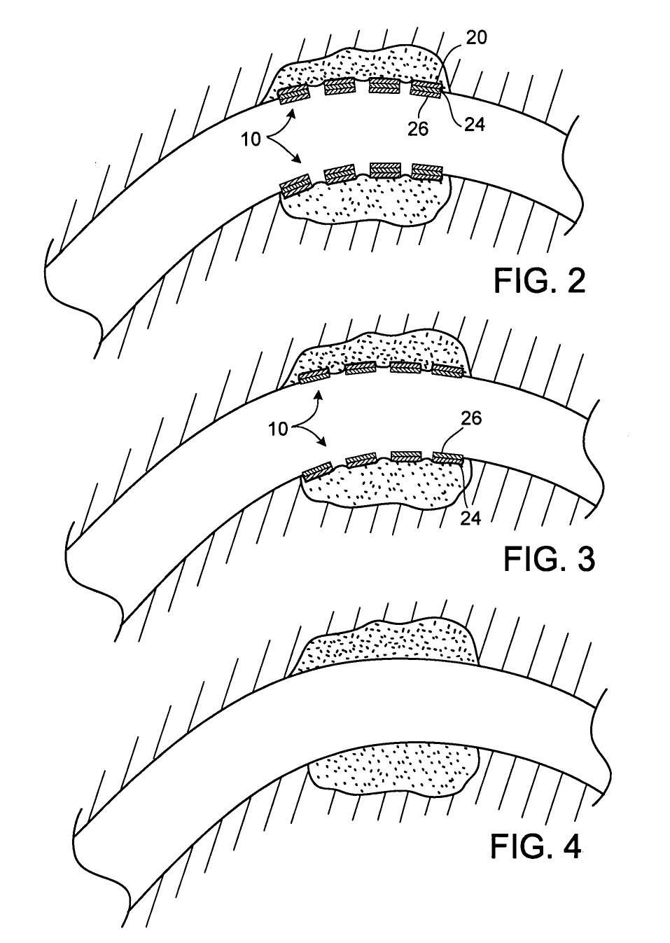

Referring to FIGS. 2-4, this arrangement of outer, middle, and inner portions 20, 24, 26 can provide a stent which selectively erodes from outside in (e.g., from the walls towards the center of the vessel in which the stent is implanted). In other embodiments, stents can be constructed with portions or layers having erosion rates that increase towards the walls of the vessels to provide stents which selectively erode from the inside out. Although the illustrative embodiment includes three portions (e.g., layers), stents can be constructed with two or more portions as is appropriate for a particular application. Similarly, although the illustrated embodiment is substantially uniform along the length of stent 10, some embodiments include portions 20, 24, 26 which are varied along a direction (e.g., length) of a stent to allow the stent to erode in a predetermined sequence. For example, in some embodiments, the thicknesses of the portions 20, 24, and 26 can be varied relative to each other with inner portion 26

Portions 20, 24, and 26 can have the same chemical composition or different compositions. For example, inner portion 26 may contact bodily fluid more than outer portion 20 (which may contact the wall of the body passageway), and as a result, the inner portion may erode more quickly than the outer portion. To compensate for the difference in erosion and to allow a given cross section of stent 28 to erode relatively uniformly from portions 20, 26 to middle portion 24, the inner portion may have a chemical composition, molecular weight, or cross-linking that erodes more slowly than the chemical composition, molecular weight, or cross-linking of the outer portion.

Embodiments of the stents can include (e.g., be made from) a biocompatible material capable of eroding within the body. The erodible or bioerodible material can be a substantially pure metallic element or an alloy. Examples of metallic elements include iron and magnesium. Examples of alloys include iron alloys having, by weight, 88-99.8% iron, 0.1-7% chromium, 0-3.5% nickel, and less than 5% of other elements (e.g., magnesium and/or zinc); or 90-96% iron, 3-6% chromium and 0-3% nickel plus 0-5% other metals. Other examples of alloys include magnesium alloys, such as, by weight, 50-98% magnesium, 0-40% lithium, 0-5% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2%-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Magnesium alloys are also available under the names AZ91D, AM50A, and AE42. Other erodible materials are described in Bolz, U.S. Pat. No. 6,287,332 (e.g., zinc-titanium alloy and sodium-magnesium alloys); Heublein, U.S. Patent Application 2002000406; and Park, Science and Technology of Advanced Materials, 2, 73-78 (2001), all of which are hereby incorporated by reference herein in their entirety. In particular, Park describes Mg—X—Ca alloys, e.g., Mg—Al—Si—Ca, Mg—Zn—Ca alloys.

Portions of tubular body 13 with reduced erosion rates can include an erodible combination of the erodible material as described above and one or more first materials capable of changing (e.g., reducing) the erosion rate of the erodible material. In some embodiments, the erosion rate of a first portion (e.g., inner portion 26) of stent 10 is from about 10% to about 300% less than (i.e., 1.1 to 3 times slower than) the erosion rate of a second portion (e.g., outer portion 20), for example, from about 25% to about 200% less, or from about 50% to about 150% less. The erosion rate of a portion can range from about 0.01 percent of an initial mass of that portion per day to about 1 percent of the initial mass of that portion per day, e.g., from about 0.1 percent of the initial mass of that portion per day to about 0.5 percent of the initial mass of that portion per day. Examples of first materials include magnesium nitride, magnesium oxide, magnesium fluoride, iron nitride and iron carbide. Iron nitride and iron carbide materials are discussed in Weber, Materials Science and Engineering, A199, 205-210 (1995), and magnesium nitride is discussed in Tian, Surface and Coatings Technology, 198, 454-458 (2005), the entire disclosure of each is hereby incorporated by reference herein.

The concentration(s) of the first material(s) in outer, middle, and/or inner portions 20, 24, 26 can vary, depending on the desired time to erode through the portions. In embodiments in which the first material(s) has a slower erosion rate than the erosion rate of the erodible material, the higher the concentration(s) of the first material(s), the more time it takes to erode through the portions. The total concentration of the first material(s) in a portion can range from about 1 percent to about fifty percent. The concentrations of first material(s) in the portions 20, 24, 26 can be the same or different. For example, to compensate for the difference in erosion between portions 20, 26 and to allow a given cross section of stent 28 to erode relatively uniformly from the portions 20, 26 to middle portion 24, the inner portion may have a higher concentration of first material(s) than the outer portion along the cross section.

The thicknesses of outer, middle, and inner portions 20, 24, 26 containing the first material(s) can also vary, depending on the desired time to erode through the portions. The thickness of an inner, a middle, or an outer portion including the first material(s) can range from about 1 nm to about 750 nm. The thicknesses of the portions 20, 24, 26 can be the same or different. For example, to compensate for the difference in erosion rates between portions 20, 26 and to allow a cross section of stent 10 to erode relatively uniformly from the portions 20, 26 to middle portion 24, the inner portion may be thicker than the outer portion along the cross section.

The combination of the first material(s) and the erodible material can be formed by plasma treatment, such as plasma immersion ion implantation (“PIII”). During PIII, one or more charged species in a plasma, such as an oxygen and/or a nitrogen plasma, are accelerated at high velocity toward a substrate, such as a stent including the erodible material (“a pre-stent”). This process is described below and in U.S. patent application Ser. No. 11/327,149 which is incorporated herein in its entirety. In some embodiments, a pre-stent can be made, for example, by forming a tube including the erodible material and laser cutting a stent pattern in the tube, or by knitting or weaving a tube from a wire or a filament including the erodible material.

In some embodiments, a PIII processing system can include a vacuum chamber having a vacuum port connected to a vacuum pump and a gas source for delivering a gas, e.g., oxygen, nitrogen, or a silane to the chamber to generate a plasma. In use, a plasma is generated in the chamber and accelerated to the pre-stent.

Acceleration of the charged species, e.g., particles, of the plasma towards a pre-stent can be driven by an electrical potential difference between the plasma and the pre-stent. Alternatively, one could also apply the electrical potential difference between the plasma and an electrode that is underneath the pre-stent such that the stent is in a line-of-sight. Such a configuration can allow part of the pre-stent to be treated, while shielding other parts of the pre-stent. This can allow for treatment of different portions of the pre-stent with different energies and/or ion densities.

In some embodiments, the potential difference can be greater than 10,000 volts, e.g., greater than 20,000 volts, greater than 40,000 volts, greater than 50,000 volts, greater than 60,000 volts, greater than 75,000 volts, or even greater than 100,000 volts. Upon impact with the surfaces of the pre-stent, the charged species, due to their high velocity, penetrate a distance into the pre-stent, react with the erodible material, and form stent having portions. The penetration depth is controlled, at least in part, by the potential difference between the plasma and the pre-stent. Consequently, both ion penetration depth and ion concentration can be modified by changing the configuration of the PIII processing system. For example, when the ions have a relatively low energy, e.g., 10,000 volts or less, penetration depth is relatively shallow when compared with the situation when the ions have a relatively high energy, e.g., greater than 40,000 volts. The dose of ions being applied to a surface can range from about 1×10⁴ ions/cm² to about 1×10⁹ ions/cm², e.g., from about 1×10⁵ ions/cm² to about 1×10⁸ ions/cm².

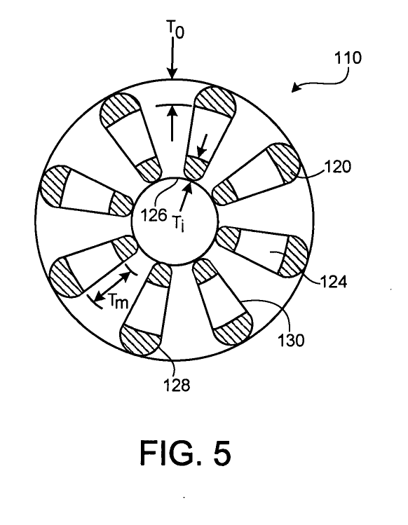

Other configurations of stents are also possible. For example, corners 28 (at which faces 30 meet) can erode more quickly than central parts of the faces as the corners are exposed on two sides. Referring to FIG. 5, a more uniform erosion rate across face 130 can be provided using a stent 110 that has outer, middle, and inner portions 120, 124, 126 with arcuate surfaces 128 joining faces 130. In some embodiments, the resulting more uniform erosion rate can limit unwanted preferential erosion of portions of the stent which may result in fragmentation of a stent. Such stents can be manufactured, for example, by forming stents and then subjecting the stents to mechanical and/or chemical polishing.

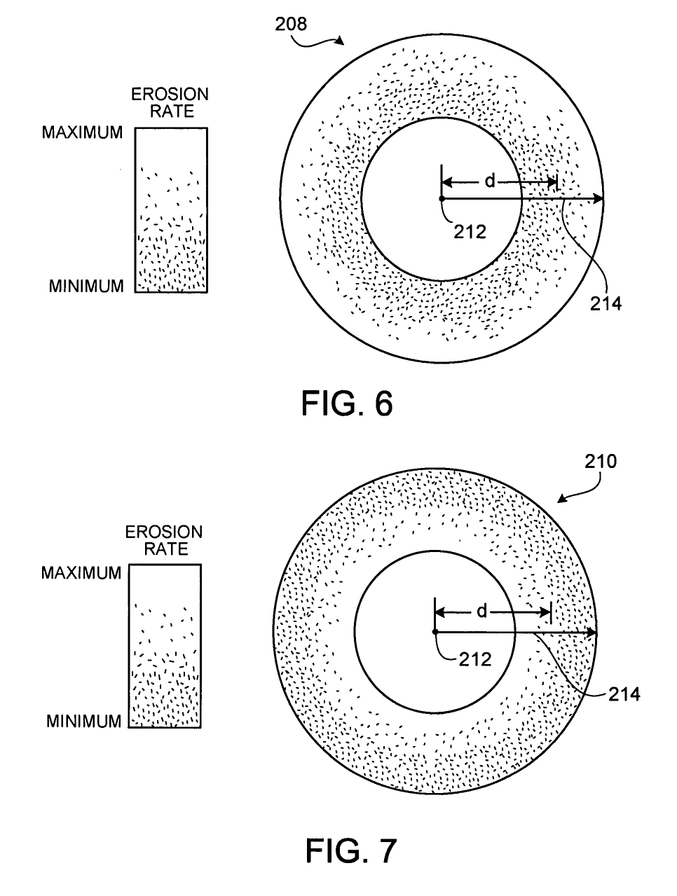

Referring to FIGS. 6 and 7, stents 208 and 210 can have local erosion rates that vary as continuous functions of radial distance d from a longitudinal axis 212 of the stents. More specifically, local erosion rates can increase (stent 208) or decrease (stent 210) with increasing distance from longitudinal axis 212 along radius 214. These continuous functions can be linear or nonlinear. Similarly, the continuous functions can be constant in direction (e.g., substantially consistently increasing (or decreasing) with increasing radial distance from longitudinal axis 212) or can vary in direction (e.g., initially increasing with increasing radial distance and then decreasing with increasing radial distance). These gradual changes in local erosion rates contrast with the changes in local erosion rates found in, for example, layered stents (e.g., see FIGS. 1 and 5 for stents with erosion profiles that would resemble a square wave). Endoprotheses with gradual changes to their rate of decomposition or erosion can be easier to produce than endoprotheses with specific zones of decomposition.

Stents with gradually varying local erosion rates can be manufactured from sheets (e.g., sheets including metals and/or metal alloys or polymer sheets) with bioerosion rates that vary with depth. In one example, polymers whose bioerosion rates decrease with the degree of cross-linking can be exposed to ion bombardment on one side to produce a degree of cross-linking that decreases with distance from the side on which the sheet is exposed to ion bombardment. The edges of the polymer sheet can then be attached to each other to form a tubular member from which a stent is manufactured as described in more detail in U.S. patent application Ser. No. 10/683,314, filed Oct. 10, 2003; and U.S. patent application Ser. No. 10/958,435, filed Oct. 5, 2004, incorporated herein by reference above. In another example, a metal sheet can be formed of a magnesium alloy containing magnesium nitride with the percentage of magnesium nitride varying with distance from a broad side of the sheet.

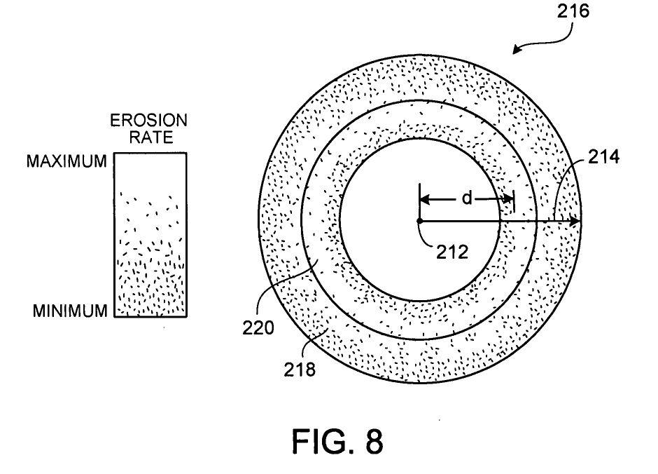

The direction of the changes in the local erosion rate can be controlled by how the sheet is rolled to join the edges. For example, referring to FIG. 6, rolling a sheet with the more less erodible side on the interior can produce a tubular member for formation of stent 208 with local erosion rates that increase with increasing radial distance d from axis 212. Similarly, referring to FIG. 7, rolling a sheet with the less erodible side on the exterior can produce a tubular member for formation of stent 210 with local erosion rates that decrease with increasing radial distance d from the axis 212. Similar approaches can be used to form stents in which local erosion rates increase (or decrease) from both exterior and interior surface of the stents towards the middle of the stent. For example, referring to FIG. 8, two sheets 218, 220 can be joined along their more erodible sides before the combined sheet is rolled to form a tubular member for formation of a stent 216 in which local erosion rates increase from both the interior and exterior surfaces of the stent towards the center of the stent. Stents with erosion rates that increase with increasing distance from stent surfaces can be initially resistant to erosion (e.g., while the body lumen reestablishes its own patentcy) and then quickly erode without fragmenting

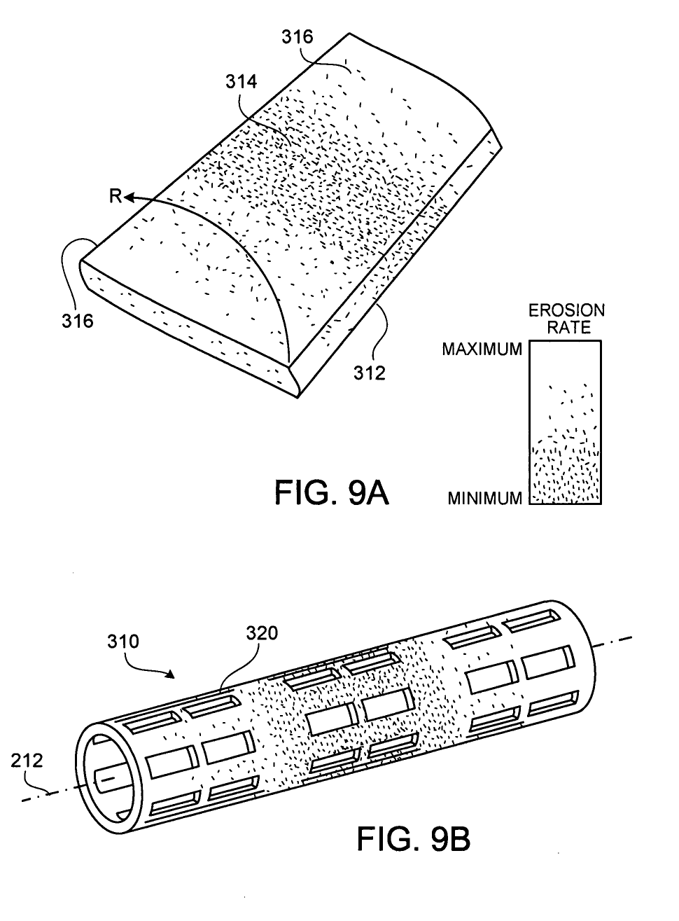

Referring to FIGS. 9A and 9B, stents 310 can also have local erosion rates that vary along longitudinal axis 212. Longitudinal variations in local erosion rates can be in place of or in addition to radial variations in local erosion rates. As with radial variations, longitudinal variations in local erosion rates can be continuous or discontinuous functions. For example, in some embodiments, a polymer sheet 312 can be exposed to ion bombardment in a manner to cause a higher relative degree of cross-linking and lower local erosion rates in a central region 314 of the polymer sheet and lower relative degrees of cross-linking and lower local erosion rates in end regions 316 of the polymer sheet. Polymer sheet 312 can be rolled (see arrow R) to form a tubular member from which stent 310 is formed as described above. Resulting stent 310 has local erosion rates that decrease towards a central section 318 of the stent. Thus, stent 310 tends to erodes from end sections 320 towards middle section 318. In some embodiments, longitudinal sections of stents can be formed from different materials to provide desired longitudinal variations in local erosion rates. For example, a stent could be formed with a center section and two end sections including a magnesium alloy. The center section can include a greater proportion of a corrosion resistant material (e.g., magnesium nitride) such that the two end sections erode more quickly than the center section.

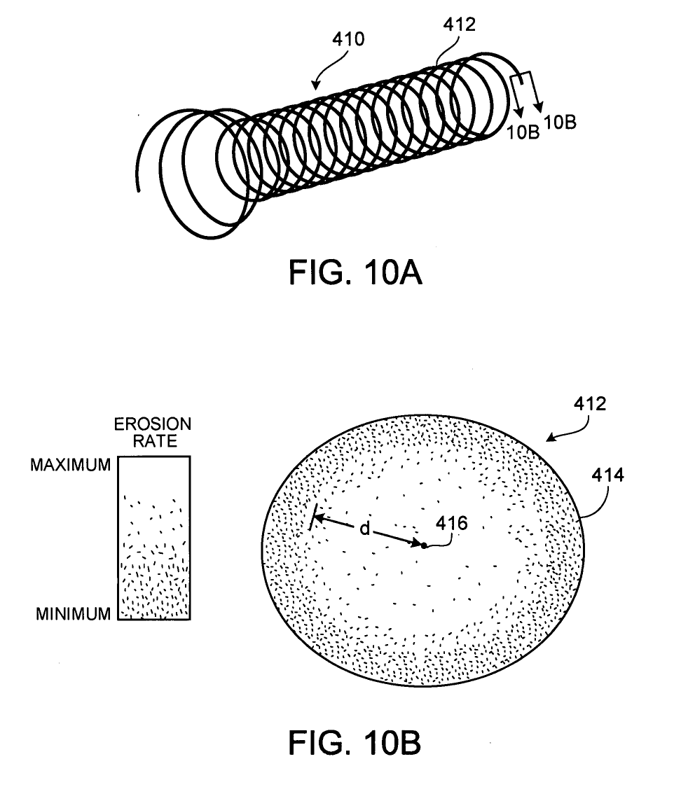

Referring to FIGS. 10A and 10B, stent 410 can include a bioerodible member 412 (e.g., a wire or fiber) having a solid cross-section with an arcuate outer surface 414. As used herein, solid denotes an object that is not hollow. In some embodiments, bioerodible member 412 is substantially round (e.g., having a width to height aspect ratio of 0.95:1 to 1.05:1). Bioerodible member 412 can include (e.g., be formed of) the materials described in elsewhere herein (e.g., bioerodible metals and/or polymers). In some embodiments, bioerodible member 412 can be formed of a polymer which has a degree of cross-linking that increase with radial distance d from a longitudinal axis 416 of the bioerodible member. Thus, member 412 initially erodes slowly to substantially maintain the structural stability of stent 410. Then, as the more highly cross-linked outer portions of stent 410 erode away, the rate of bioerosion increases as less highly cross-linked portions of the stent are exposed.

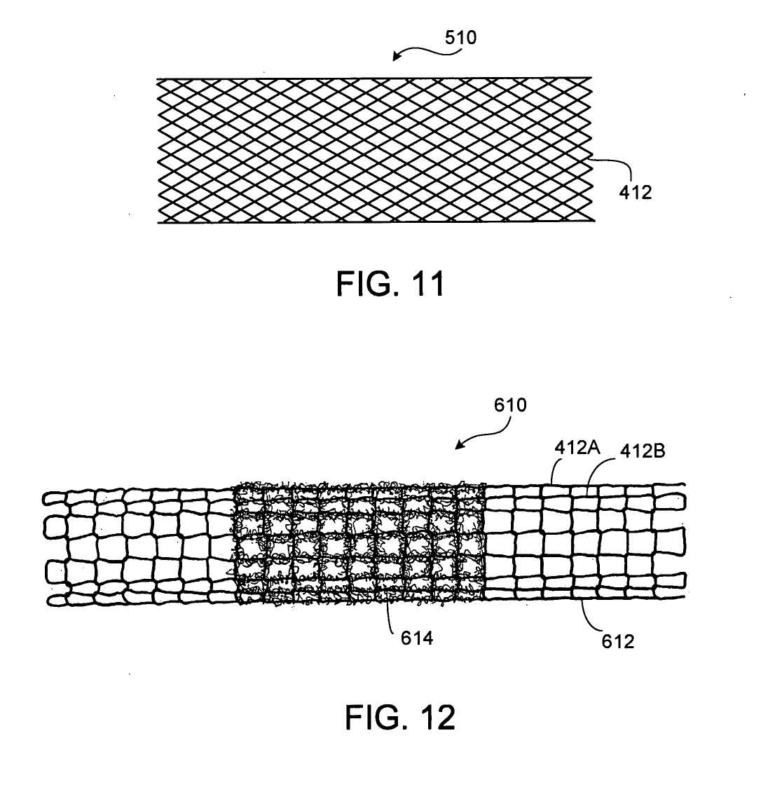

Referring to FIG. 10A, in some embodiments, stents with bioerodible members can be formed of a single longitudinally extending bioerodible member 412 (e.g., as a coiled wire stent 410). Referring to FIGS. 11 and 12, in some embodiments, stents with bioerodible members can be formed of multiple bioerodible members attached together (e.g., woven stents 510, 610). Woven stents and their manufacture are discussed in more detail in U.S. Pat. Nos. 5,824,077 and 5,674,276, which are incorporated herein in their entirety. In stents 510, 610, formed of multiple bioerodible members 412, individual bioerodible members 412A and 412B can have different erosion rates. This is another approach to forming stents which selectively degrade in a particular sequence. Referring to FIG. 12 for example, loops near end sections 612 can have higher erosion rates than loops in middle section 614 such that stent 610 tends to degrade from the ends towards the middle.

In use, the stents can be used, e.g., delivered and expanded, using a catheter delivery system, such as a balloon catheter system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Radius® or Symbiot® systems, available from Boston Scientific Scimed, Maple Grove, Minn.

The stents described herein can be of a desired shape and size (e.g., coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stents can be balloon-expandable, or a combination of self-expandable and balloon-expandable (e.g., as described in U.S. Pat. No. 5,366,504).

While a number of embodiments have been described above, the invention is not so limited.

The stents described herein can include non-metallic structural portions, e.g., polymeric portions. The polymeric portions can be erodible. The polymeric portions can be formed from a polymeric blend. The stents described herein can be a part of a covered stent or a stent-graft. For example, a stent can include and/or be attached to a biocompatible, non-porous or semi-porous polymer matrix including polytetrafluoroethylene (PTFE), expanded PTFE, polyethylene, urethane, or polypropylene. Other exemplary polymers include, for example, polynorbomene, polycaprolactone, polyenes, nylons, polycyclooctene (PCO), blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, trans-isoprene, blends of polycaprolactone and n-butylacrylate and blends thereof. Polymeric stents have been described in U.S. patent application Ser. No. 10/683,314, filed Oct. 10, 2003; and U.S. patent application Ser. No. 10/958,435, filed Oct. 5, 2004, the entire contents of each is hereby incorporated by reference herein. The erosion rate of stent portions including bioerodible polymers can be reduced, for example, by increased cross-linking of the polymers. The cross-linking of the polymers can be increased by, for example, ion bombardment of the polymer before, during, or after manufacture of a stent.

As an example, in some embodiments, the corrosion rate of a bioerodible material can be increased by addition of one or more other materials. As an example, outer and middle portions 20, 24 of tubular body 13 can include an erodible combination of the erodible material of inner portion 26 and one or more first materials capable of increasing the erosion rate. For example, inner portion 26 can be formed of iron, and middle and outer portions 24, 20 can be formed of alloys of iron and platinum.

In some embodiments, bioerodible stents can be formed of materials chosen such that the stent is structurally stable (e.g., capable of maintaining patentcy of a body lumen) for at least 30 days before significantly biodegrading.

The stents described herein can have non-circular transverse cross-sections. For example, transverse cross-sections can be polygonal, e.g., square, hexagonal or octagonal.

The stents can include a releasable therapeutic agent, drug, or a pharmaceutically active compound, such as described in U.S. Pat. No. 5,674,242, U.S. Ser. No. 09/895,415, filed Jul. 2, 2001, U.S. Ser. No. 11/111,509, filed Apr. 21, 2005, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics. The therapeutic agent, drug, or a pharmaceutically active compound can be dispersed in a polymeric coating carried by the stent. The polymeric coating can include more than a single layer. For example, the coating can include two layers, three layers or more layers, e.g., five layers. The therapeutic agent can be a genetic therapeutic agent, a non-genetic therapeutic agent, or cells. Therapeutic agents can be used singularly, or in combination. Therapeutic agents can be, for example, nonionic, or they may be anionic and/or cationic in nature. An example of a therapeutic agent is one that inhibits restenosis, such as paclitaxel. The therapeutic agent can also be used, e.g., to treat and/or inhibit pain, encrustation of the stent or sclerosing or necrosing of a treated lumen. Any of the above coatings and/or polymeric portions can by dyed or rendered radio-opaque.

The stents described herein can be configured for non-vascular lumens. For example, it can be configured for use in the esophagus or the prostate. Other lumens include biliary lumens, hepatic lumens, pancreatic lumens, uretheral lumens and ureteral lumens.

In some embodiments, a stent can be produced from a metallic pre-stent. During production, all portions of the pre-stent are implanted with a selected species, e.g., oxygen or nitrogen. After a desired implantation time, all exposed surfaces of a selected segment of implanted pre-stent are covered with a coating, e.g., a protective polymeric coating, such as a styrene-isoprene-butadiene-styrene (SIBS) polymer, to produce a coated pre-stent. Coated pre-stent is then implanted with a desired species for the desired time. Conditions for implantation are selected to penetrate the desired species more deeply into the except where the coating protects the selected segment from additional implantation by the desired species. At this point, the coating can be removed, e.g., by rinsing with a solvent such as toluene, to complete the production of the stent. Similarly, a stent having tapered thicknesses can be produced by masking the interior and/or outer portions with a movable sleeve and longitudinally moving the sleeve and/or the stent relative to each other during implantation.

Other methods of making a stent are also possible. For example, an tube including a bioerodible material can be extruded and then processed to form a stent.

Other embodiments are within the scope of the claims.

What is claimed is:

1. An endoprothesis comprising a bioerodible body having local erosion rates of the body that vary as a continuous function of radial distance from the longitudinal axis.

2. The endoprothesis of claim 1 wherein a first portion of the body has a first erosion rate and a second portion of the body has a second erosion rate that is greater than the first erosion rate and the distance between the second portion of the body and the longitudinal axis is greater than the distance between the first portion of the body and the longitudinal axis.

3. The endoprothesis of claim 1 wherein a first portion of the body has a first erosion rate and a second portion of the body has a second erosion rate that is less than the first erosion rate and the distance between the second portion of the body and the longitudinal axis is greater than the distance between the first portion of the body and the longitudinal axis.

4. The endoprothesis of claim 1 wherein the endoprosthesis defines a tubular lumen parallel to the longitudinal axis.

5. The endoprothesis of claim 1 wherein the body comprises a polymer.

6. The endoprothesis of claim 5 wherein the body comprises a cross-linkable polymer that has a degree of cross-linking that varies as a function of radial distance from the longitudinal axis.

7. The endoprothesis of claim 1 wherein the body comprises at least one metal.

8. The endoprothesis of claim 7 wherein the body further comprises at least one polymer.

9. The endoprothesis of claim 1 wherein a first erosion rate of a first portion of the body is between about 1 and 3 percent of the mass of the first portion per day.

10. The endoprothesis of claim 9 wherein a second erosion rate of a second portion of the body is between about 0.1 and 1 percent of the mass of the second portion per day.

11. The endoprothesis of claim 1 comprising a stent.

12. An endoprothesis comprising a bioerodible member having a solid cross-section with an arcuate outer surface.

13. The endoprothesis of claim 12 wherein the bioerodible member comprises a substantially round portion.

14. The endoprothesis of claim 13 further comprising a plurality of bioerodible members attached together, each of the bioerodible members having substantially round solid cross-sections.

15. The endoprothesis of claim 14 wherein the bioerodible members comprise wire.

16. The endoprothesis of claim 12 wherein the outer surface of the bioerodible member comprises flat faces joined by radiused transition sections.

17. The endoprothesis of claim 12 wherein an erosion rate of the bioerodible member is between about 1 and 3 percent of the mass of the bioerodible member per day.

18. The endoprothesis of claim 17 wherein an erosion rate of the bioerodible member is between about 0.1 and 1 percent of the mass of the bioerodible member per day.

19. The endoprothesis of claim 12 comprising a stent.

20. The endoprothesis of claim 12 wherein the endoprosthesis defines a tubular lumen parallel to a longitudinal axis.

21. An endoprothesis comprising a body having local erosion rates that vary along a first direction, and that vary along a second direction.

22. The endoprothesis of claim 21 wherein the endoprosthesis has a longitudinal axis, the first direction is transverse to the longitudinal axis, and the second direction is along the longitudinal direction.
